(19) United States
(12) Patent Application Publication
(10) Pub. No.: US 2022/0411439 A1
(43) Pub. Date: Dec. 29, 2022

(54) POLYMORPHS OF A MACROCYCLIC KINASE INHIBITOR

(71) Applicant: TURNING POINT THERAPEUTICS, INC., San Diego, CA (US)

(72) Inventors: Wei DENG, San Diego, CA (US); Evan W. ROGERS, San Diego, CA (US); Yuelie LU, San Diego, CA (US); Han ZHANG, San Diego, CA (US); Jing LIU, San Diego, CA (US)

(21) Appl. No.: 17/618,060

(22) PCT Filed: Jun. 16, 2020
(86) PCT No.: PCT/US20/37892

§ 371 Date: Dec. 10, 2021

Related U.S. Application Data
(60) Provisional application No. 62/863,493, filed on Jun. 19, 2019.
(60) Provisional application No. 62/959,940, filed on Jan. 11, 2020.
(60) Provisional application No. 63/036,102, filed on Jun. 8, 2020.

(51) Int. Cl. C07D 498/22; A61P 35/00

(52) U.S. Cl. CPC C07D 498/22; A61P 35/00; C07B 2200/13

(57) ABSTRACT

This disclosure relates to polymorphs of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one that are useful in the treatment of disease, such as cancer, in mammals. This disclosure also relates to compositions including such polymorphs, and to methods of using such compositions in the treatment of diseases, such as cancer, in mammals, especially in humans.

### CROSS-REFERENCE TO RELATED APPLICATIONS

This application claims priority under 35 U.S.C. § 119(e) to U.S. Provisional Application Ser. No. 62/863,493 filed on Jun. 19, 2019, U.S. Provisional Application Ser. No. 62/959,940 filed on Jan. 11, 2020, and, U.S. Provisional Application Ser. No. 63/036,102 filed on Jun. 8, 2020 the entire disclosures of which are incorporated herein by reference.

### TECHNICAL FIELD

This disclosure relates to polymorphs of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one, or a hydrate thereof, that are useful in the treatment of disease, such as cancer, in mammals. This disclosure also relates to compositions including such polymorphs, and to methods of using such compositions in the treatment of diseases, such as cancer, in mammals, especially in humans.

### BACKGROUND

The compound (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one (also herein referred to as “Compound I”) represented by the formula I

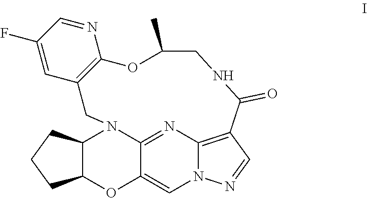 [SMILES: C[C@H]1CNC(=O)c2cnn3cc4c(nc23)N(Cc2cc(F)cnc2O1)[C@@H]1CCC[C@@H]1O4]

is a potent small-molecule multi-target kinase inhibitor showing activity against wild-type and mutant RET, SRC and FGFR1/2. Compound I has properties, including anti-tumor properties, that are pharmacologically mediated through inhibition of tyrosine kinase receptors. Compound I is disclosed in International Patent Publication No. WO 2019/126121 A1 (International Patent Application No. PCT/US2018/066158), filed Dec. 18, 2018, which is incorporated herein by reference in its entirety.

Protein kinases are key regulators for cell growth, proliferation and survival. A variety of diseases, such as cancer, pain, neurological diseases, autoimmune diseases, and inflammation, have been shown to be mediated by receptor tyrosine kinases, such as activating mutated RET or FGFR1/2. For example, genetic and epigenetic alterations can accumulate in cancer cells leading to abnormal activation of signal transduction pathways which drive malignant processes. Manning, G. et al., Science 2002, 298, 1912-1934. Pharmacological inhibition of these signaling pathways presents promising intervention opportunities for targeted cancer therapies. Sawyers, C., Nature 2004, 432, 294-297.

While Compound I has found application in treating disease associated with receptor tyrosine kinases, such as RET activating mutations, it is advantageous to have polymorphic forms having improved properties, such as improved crystallinity, dissolution properties, and/or decreased hygroscopicity, while maintaining chemical and enantiomeric stability properties.

### SUMMARY

In one aspect, the present disclosure provides a crystalline form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one, or a hydrate thereof.

In another embodiment, the crystalline polymorph form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one is a hydrate.

In another embodiment, the crystalline polymorph form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one is anhydrous.

In another embodiment, the crystalline polymorph form A or D of compound I can be represented by the formula

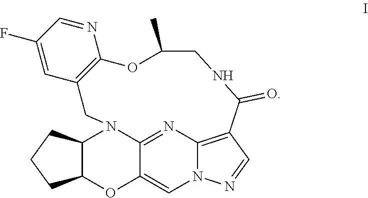 [SMILES: C[C@H]1CNC(=O)c2cnn3cc4c(nc23)N(Cc2cc(F)cnc2O1)[C@@H]1CCC[C@@H]1O4]

In a further embodiment, the crystalline polymorph form has a powder X-ray diffraction pattern comprising a peak at diffraction angle (2θ) of 20.1±0.1. In a further embodiment, the crystalline polymorph form has a powder X-ray diffraction pattern comprising peaks at diffraction angles (2θ) of 11.3±0.1 and 20.1±0.1. In a further embodiment, the crystalline polymorph form has a powder X-ray diffraction pattern comprising peaks at diffraction angles (2θ) of 11.3±0.1, 20.1±0.1, and 23.9±0.1. In a further embodiment, the crystalline polymorph form has a powder X-ray diffraction pattern comprising peaks at diffraction angles (2θ) of 11.3±0.1, 18.0±0.1, 20.1±0.1, and 23.9±0.1. In a further embodiment, the crystalline polymorph form has a powder X-ray diffraction pattern comprising peaks at diffraction angles (2θ) of 10.3±0.1, 11.3±0.1, 18.0±0.1, 20.1±0.1, and 23.9±0.1. In a further embodiment, the crystalline polymorph form has a powder X-ray diffraction pattern comprising peaks at diffraction angles (2θ) of 10.3±0.1, 11.3±0.1, 16.6±0.1, 18.0±0.1, 20.1±0.1, and 23.9±0.1. In a further embodiment, the crystalline polymorph form has a powder X-ray diffraction pattern comprising peaks at diffraction angles (2θ) of 10.3±0.1, 11.3±0.1, 16.6±0.1, 18.0±0.1, 20.1±0.1, 20.5±0.1, and 23.9±0.1.

In a further aspect the crystalline form has a powder X-ray diffraction pattern comprising peaks at diffraction angles (2θ) essentially the same as shown in FIG. 1.

In a further embodiment, the crystalline polymorph form has a powder X-ray diffraction pattern comprising a peak at diffraction angle (2θ) of 18.8±0.1. In a further embodiment, the crystalline polymorph form has a powder X-ray diffraction pattern comprising peaks at diffraction angles (2θ) of 18.8±0.1 and 20.9±0.1. In a further embodiment, the crystalline polymorph form has a powder X-ray diffraction pattern comprising peaks at diffraction angles (2θ) of 12.3±0.1, 18.8±0.1, and 20.9±0.1. In a further embodiment, the crystalline polymorph form has a powder X-ray diffraction pattern comprising peaks at diffraction angles (2θ) of 12.3±0.1, 18.8±0.1, 19.4±0.1, and 20.9±0.1. In a further embodiment, the crystalline polymorph form has a powder X-ray diffraction pattern comprising peaks at diffraction angles (2θ) of 12.3±0.1, 13.1±0.1, 18.8±0.1, 19.4±0.1, and 20.9±0.1. In a further embodiment, the crystalline polymorph form has a powder X-ray diffraction pattern comprising peaks at diffraction angles (2θ) of 12.3±0.1, 13.1±0.1, 15.3±0.1, 18.8±0.1, 19.4±0.1, and 20.9±0.1. In a further embodiment, the crystalline polymorph form has a powder X-ray diffraction pattern comprising peaks at diffraction angles (2θ) of 9.4±0.1, 12.3±0.1, 13.1±0.1, 15.3±0.1, 18.8±0.1, 19.4±0.1, and 20.9±0.1.

In a further aspect the crystalline form has a powder X-ray diffraction pattern comprising peaks at diffraction angles (2θ) essentially the same as shown in FIG. 3.

The present disclosure further provides a pharmaceutical composition comprising a polymorph form A or form D of Compound I of the formula

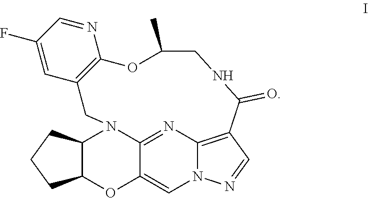 [SMILES: C[C@H]1CNC(=O)c2cnn3cc4c(nc23)N(Cc2cc(F)cnc2O1)[C@@H]1CCC[C@@H]1O4]

The present disclosure further provides a capsule comprising pharmaceutical compositions as described herein.

In another aspect, the disclosure provides a method of treating disease, especially cancer, in a mammal, including a human, the method comprising administering to the mammal a therapeutically effective amount of polymorph form A or D of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one, as described herein, or a pharmaceutical composition comprising polymorph form A or D of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one, as described herein.

In one embodiment, the present disclosure provides a method of treating abnormal cell growth in a mammal, including a human, in need of such treatment comprising, administering to said mammal a therapeutically effective amount of the free base polymorph form A or D of Compound I. In some embodiments, the abnormal cell growth is mediated by at least one genetically altered tyrosine kinase.

In some embodiments, the abnormal cell growth is mediated by RET, SRC, FGFR1/2 or a combination thereof. In some embodiments, the abnormal cell growth is mediated by wild-type or mutant RET. In some embodiments, the abnormal cell growth is mediated by wild-type or mutant SRC. In some embodiments, the abnormal cell growth is mediated by wild-type or mutant FGFR1/2.

In some embodiments, the present disclosure provides a method of treating abnormal cell growth in a patient in need of such treatment comprising, administering a therapeutically effective amount of Compound I. In some embodiments, the present disclosure provides a method of treating abnormal cell growth in a patient in need of such treatment comprising, administering a therapeutically effective amount of the free base polymorph form A or D of Compound I. In some embodiments, the abnormal cell growth is cancer mediated by a genetically altered RET. In some embodiments, the abnormal cell growth is cancer mediated by a genetically altered RET comprising at least one point mutation selected from the group consisting of A883F, E762Q, G691S, L790F, M918T, R749T, R813Q, S891A, S904A, S904F, V778I, V804L, V804M, Y791F, and Y806H.

In some embodiments, the present disclosure provides a method of treating abnormal cell growth in a patient in need of such treatment comprising, administering a therapeutically effective amount of Compound I. In some embodiments, the present disclosure provides a method of treating abnormal cell growth in a patient in need of such treatment comprising, administering a therapeutically effective amount of the free base polymorph form A or D of Compound I. In some embodiments, the abnormal cell growth is cancer mediated by a fusion protein comprising a fragment of a protein encoded by an RET gene and a fragment of a protein which can form coiled-coil interaction to facilitate the protein dimerization or oligomerization. In some embodiments, the abnormal cell growth is cancer mediated by a fusion protein comprising a fragment of a protein encoded by an RET gene and a fragment of a protein encoded by a gene selected from the group consisting of KIF5B, CCDC6, NCOA4, TRIM24, TRIM33, PRKAR1A, GOLGA5, KTN1, ERC1, MBD1, and TRIM27. In some embodiments, the fusion protein comprises a fragment of a protein encoded by an RET gene and a fragment of a protein encoded by a KIF5B gene. In some embodiments, the genetically altered RET is a KIF5B-RET, CCDC6-RET, NCOA4-RET, TRIM24-RET, TRIM33-RET, PRKAR1A-RET, GOLGA5-RET, KTN1-RET, ERC1-RET, MBD1-RET, or TRIM27-RET fusion protein. In some embodiments, the genetically altered RET is a KIF5B-RET fusion protein. In some embodiments, the KIF5B-RET fusion protein is a wild-type protein. In some embodiments, the KIF5B-RET fusion protein comprises at least one resistance mutation. In some embodiments, the KIF5B-RET fusion protein comprises at least one gate keeper resistance mutation. In some embodiments, the KIF5B-RET fusion protein comprises at least one solvent front resistance mutation. In some embodiments, the KIF5B-RET fusion protein comprises at least one mutation selected from the group consisting of A883F, E762Q, G691S, L790F, M918T, R749T, R813Q, S891A, S904A, S904F, V778I, V804L, V804M, Y791F, Y806H, V804M, G810R, G810C, and G810S. In some embodiments, the KIF5B-RET fusion protein comprises at least one mutation selected from the group consisting of V804M, G810R, G810C, and G810S.

In some embodiments, the abnormal cell growth is cancer. In some embodiments, the cancer is selected from the group consisting of lung cancer, non-small cell lung cancer, small cell lung cancer, bone cancer, pancreatic cancer, skin cancer, cancer of the head or neck, hepatocellular carcinoma, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, gastric and esophago-gastric cancers, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, such as anaplastic large cell lymphoma, cancer of the bladder, cancer of the kidney or ureter, renal cell carcinoma, carcinoma of the renal pelvis, neoplasms of the central nervous system (CNS), glioblastoma, primary CNS lymphoma, spinal axis tumors, brain stem glioma, pituitary adenoma, inflammatory myofibroblastic tumors, and combinations thereof.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A crystalline polymorph form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one, or a hydrate thereof.

2. The crystalline polymorph form of clause 1, wherein the crystalline form is a polymorph form of the free base of (7S,13R)-11-fluoro-7,13-dimethyl-6,7,13,14-tetrahydro-1,15-ethenopyrazolo[4,3-f][1,4,8,10]benzoxatriazacyclotridecin-4(5H)-one, or a hydrate thereof.

3. The crystalline polymorph of clause 1 or 2, wherein the crystalline form is a monohydrate.

4. The crystalline polymorph of clause 1 or 2, wherein the crystalline form is anhydrous.

5. A crystalline polymorph form A of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one having a powder X-ray diffraction pattern comprising a peak at diffraction angle (2θ) of 20.1±0.1.

6. The crystalline polymorph form of clause 5, wherein the crystalline polymorph form has a powder X-ray diffraction pattern comprising a peak at diffraction angle (2θ) of 11.3±0.1 and 20.1±0.1.

7. The crystalline polymorph form of clause 5 or 6, wherein the crystalline polymorph form has a powder X-ray diffraction pattern comprising a peak at diffraction angle (2θ) of 11.3±0.1, 20.1±0.1, and 23.9±0.1.

8. The crystalline polymorph form of any one of clauses 5 to 7, wherein the crystalline polymorph form has a powder X-ray diffraction pattern comprising a peak at diffraction angle (2θ) of 11.3±0.1, 18.0±0.1, 20.1±0.1, and 23.9±0.1.

9. The crystalline polymorph form of any one of clauses 5 to 8, wherein the crystalline polymorph form has a powder X-ray diffraction pattern comprising a peak at diffraction angle (2θ) of 10.3±0.1, 11.3±0.1, 18.0±0.1, 20.1±0.1, and 23.9±0.1.

10. The crystalline polymorph form of any one of clauses 5 to 9, wherein the crystalline polymorph form has a powder X-ray diffraction pattern comprising a peak at diffraction angle (2θ) of 10.3±0.1, 11.3±0.1, 16.6±0.1, 18.0±0.1, 20.1±0.1, and 23.9±0.1.

11. The crystalline polymorph form of any one of clauses 5 to 10, wherein the crystalline polymorph form has a powder X-ray diffraction pattern comprising a peak at diffraction angle (2θ) of 10.3±0.1, 11.3±0.1, 16.6±0.1, 18.0±0.1, 20.1±0.1, 20.5±0.1, and 23.9±0.1.

12. A crystalline polymorph form D of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one having, a powder X-ray diffraction pattern comprising a peak at diffraction angle (2θ) of 18.8±0.1.

13. The crystalline polymorph form of clause 12, wherein the crystalline polymorph form has a powder X-ray diffraction pattern comprising a peak at diffraction angle (2θ) of 18.8±0.1 and 20.9±0.1.

14. The crystalline polymorph form of clause 12 or 13, wherein the crystalline polymorph form has a powder X-ray diffraction pattern comprising a peak at diffraction angle (2θ) of 12.3±0.1, 18.8±0.1, and 20.9±0.1.

15. The crystalline polymorph form of any one of clauses 12 to 14, wherein the crystalline polymorph form has a powder X-ray diffraction pattern comprising a peak at diffraction angle (2θ) of 12.3±0.1, 18.8±0.1, 19.4±0.1, and 20.9±0.1.

16. The crystalline polymorph form of any one of clauses 12 to 15, wherein the crystalline polymorph form has a powder X-ray diffraction pattern comprising a peak at diffraction angle (2θ) of 12.3±0.1, 13.1±0.1, 18.8±0.1, 19.4±0.1, and 20.9±0.1.

17. The crystalline polymorph form of any one of clauses 12 to 16, wherein the crystalline polymorph form has a powder X-ray diffraction pattern comprising a peak at diffraction angle (2θ) of 12.3±0.1, 13.1±0.1, 15.3±0.1, 18.8±0.1, 19.4±0.1, and 20.9±0.1.

18. The crystalline polymorph form of any one of clauses 12 to 17, wherein the crystalline polymorph form has a powder X-ray diffraction pattern comprising a peak at diffraction angle (2θ) of 9.4±0.1, 12.3±0.1, 13.1±0.1, 15.3±0.1, 18.8±0.1, 19.4±0.1, and 20.9±0.1.

19. A crystalline polymorph form of any of any one of clauses 1 to 11, having a Raman spectra comprising at least one, at least two, at least three, at least four, at least five, at least six, at least seven, or at least about 8 peaks selected from Table 3.

20. A crystalline polymorph form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one having a powder X-ray diffraction pattern substantially the same as shown in FIG. 1.

21. A crystalline polymorph form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one having a powder X-ray diffraction pattern substantially the same as shown in FIG. 3.

22. A pharmaceutical composition comprising the crystalline polymorph form of any one of the preceding clauses.

23. A method of treating disease in a mammal, the method comprising administering to the mammal a therapeutically effective amount of the crystalline polymorph form of any one of clauses 1 to 21.

24. The method of clause 23, wherein the mammal is a human.

25. The method of clause 23 or 24, wherein the disease is selected from the group consisting of cancer, pain, neurological diseases, autoimmune diseases, and inflammation.

26. The method of any one of clauses 22 to 25, wherein the disease is cancer.

27. The method of clause 26, wherein the cancer is selected from the groups consisting of lung cancer, non-small cell lung cancer, small cell lung cancer, bone cancer, pancreatic cancer, skin cancer, cancer of the head or neck, hepatocellular carcinoma, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, gastric and esophago-gastric cancers, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, such as anaplastic large cell lymphoma, cancer of the bladder, cancer of the kidney or ureter, renal cell carcinoma, carcinoma of the renal pelvis, neoplasms of the central nervous system (CNS), glioblastoma, primary CNS lymphoma, spinal axis tumors, brain stem glioma, pituitary adenoma, inflammatory myofibroblastic tumors, and combinations thereof.

28. The method of any one of clauses 23-27, wherein the disease is cancer mediated by a genetically altered RET.

29. The method of any one of clauses 23-27, wherein the disease is cancer mediated by a genetically altered RET comprising at least one point mutation selected from the group consisting of A883F, E762Q, G691S, L790F, M918T, R749T, R813Q, S891A, S904A, S904F, V778I, V804L, V804M, Y791F, and Y806H.

30. The method of any one of clauses 23-27, wherein the disease is cancer mediated by a fusion protein comprising a fragment of a protein encoded by an RET gene and a fragment of a protein which can form coiled-coil interaction to facilitate the protein dimerization or oligomerization.

31. The method of any one of clauses 23-27, wherein the disease is cancer mediated by a fusion protein comprising a fragment of a protein encoded by an RET gene and a fragment of a protein encoded by a gene selected from the group consisting of KIF5B, CCDC6, NCOA4, TRIM24, TRIM33, PRKAR1A, GOLGA5, KTN1, ERC1, MBD1, and TRIM27.

32. The method of any one of clause 31, wherein the fusion protein comprises a fragment of a protein encoded by an RET gene and a fragment of a protein encoded by a KIF5B gene.

33. The method of clause 28, wherein the genetically altered RET is a KIF5B-RET, CCDC6-RET, NCOA4-RET, TRIM24-RET, TRIM33-RET, PRKAR1A-RET, GOLGA5-RET, KTN1-RET, ERC1-RET, MBD1-RET, or TRIM27-RET fusion protein.

34. The method of clause 28 or 33, wherein the genetically altered RET is a KIF5B-RET fusion protein.

35. The method of clause 28, 33, or 34, wherein the KIF5B-RET fusion protein is a wild-type protein.

36. The method of clause 28, 33, or 34, wherein the KIF5B-RET fusion protein comprises at least one resistance mutation.

37. The method of clause 28, 33, or 34, wherein the KIF5B-RET fusion protein comprises at least one gate keeper resistance mutation.

38. The method of clause 28, 33, or 34, wherein the KIF5B-RET fusion protein comprises at least one solvent front resistance mutation.

39. The method of clause 28, 33, or 34, wherein the KIF5B-RET fusion protein comprises at least one mutation selected from the group consisting of A883F, E762Q, G691S, L790F, M918T, R749T, R813Q, S891A, S904A, S904F, V778I, V804L, V804M, Y791F, Y806H, V804M, G810R, G810C, and G810S.

40. The method of clause 28, 33, or 34, wherein the KIF5B-RET fusion protein comprises at least one mutation selected from the group consisting of V804M, G810R, G810C, and G810S.

### Definitions

As used herein, unless otherwise indicated, the term “abnormal cell growth” refers to cell growth that is independent of normal regulatory mechanisms (e.g., loss of contact inhibition).

As used herein, unless otherwise indicated, the term “treating” means reversing, alleviating, inhibiting the progress of (i.e., curative treatment), or preventing the disorder or condition to which such term applies, or one or more symptoms of such disorder or condition. The term “treatment”, as used herein, unless otherwise indicated, refers to the act of treating as “treating” as defined immediately above. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

As used herein, the term “essentially the same” with reference to X-ray diffraction peak positions means that typical peak position and intensity variability are taken into account. For example, one skilled in the art will appreciate that the peak positions (2θ) will show some inter-apparatus variability, typically as much as 0.1°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only.

### BRIEF DESCRIPTION OF THE DRAWINGS

FIG. 1 shows a powder X-ray diffraction pattern of the crystalline form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one monohydrate, polymorph form A.

FIG. 2 shows a differential scanning calorimetry (DSC) thermogram of the crystalline form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one monohydrate, polymorph form A.

FIG. 3 shows a powder X-ray diffraction pattern of the crystalline form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one (solvent-free and anhydrous), polymorph form D.

FIG. 4 shows a differential scanning calorimetry (DSC) thermogram of the crystalline form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one (solvent-free and anhydrous), polymorph form D.

FIG. 5A is a chart showing the activity of Compound I, Loxo-292, and BLU-667 in Ba/F3 KIF5B-RET cell proliferation assay. (▴) Compound I; (▪) BLU-667; (●) Loxo-292.

FIG. 5B is a chart showing the activity of Compound I, Loxo-292, and BLU-667 in Ba/F3 KIF5B-RET cell proliferation assay. (▴) Compound I; (▪) BLU-667; (●) Loxo-292.

FIG. 6A are gel images the effect of Compound I on RET-G810C phosphorylation at residue Y905 in Ba/F3 engineered cells at various concentrations in nM.

FIG. 6B are gel images the effect of LOXO-292 on RET-G810C phosphorylation at residue Y905 in Ba/F3 engineered cells at various concentrations in nM.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

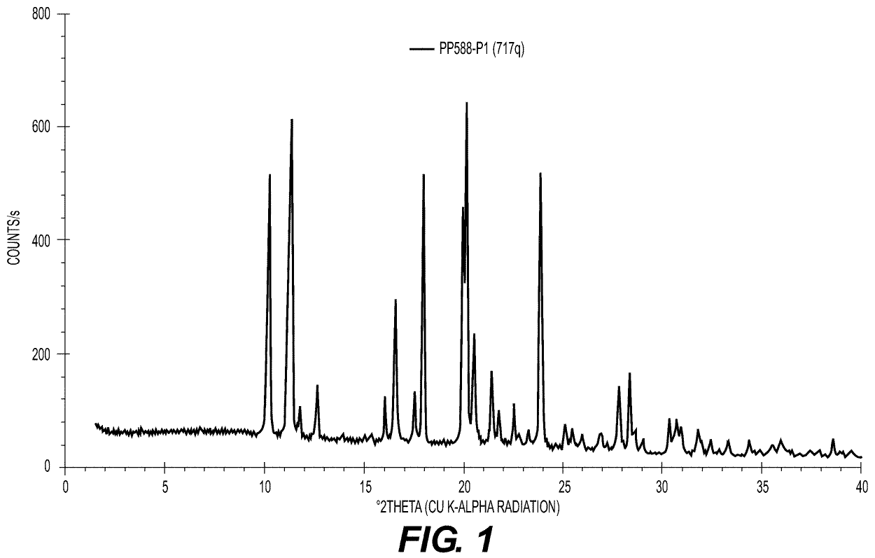

A unique physical form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one monohydrate, polymorph form A, has been prepared according to the methods described herein. The powder X-ray diffraction (PXRD) pattern of free base polymorph form A is shown in FIG. 1, with corresponding tabulated data shown in Table 1.

TABLE 1

| | | Relative | Qualitative |
|---|---|---|---|
| °2-theta | d-spacing [Å] | intensity in % | intensity* |
| 10.28 | 8.6 | 78 | vs |
| 11.33 | 7.8 | 97 | vs |
| 11.74 | 7.5 | 15 | m |
| 12.64 | 7.0 | 23 | m |
| 13.88 | 6.4 | 8 | w |
| 16.04 | 5.52 | 19 | m |
| 16.58 | 5.34 | 45 | s |
| 17.54 | 5.05 | 21 | m |
| 17.98 | 4.93 | 80 | vs |
| 19.97 | 4.44 | 72 | vs |
| 20.14 | 4.41 | 100 | vs |
| 20.51 | 4.33 | 35 | s |
| 21.43 | 4.14 | 25 | m |
| 21.78 | 4.08 | 15 | w |
| 22.04 | 4.03 | 8 | w |
| 22.52 | 3.94 | 18 | m |
| 22.78 | 3.90 | 9 | w |
| 23.27 | 3.82 | 10 | w |
| 23.88 | 3.72 | 81 | vs |
| 25.08 | 3.55 | 12 | w |
| 25.47 | 3.49 | 10 | w |
| 25.95 | 3.43 | 9 | w |
| 26.88 | 3.31 | 9 | w |
| 27.81 | 3.21 | 22 | m |
| 28.34 | 3.15 | 26 | m |
| 29.00 | 3.08 | 8 | w |
| 30.33 | 2.94 | 13 | w |
| 30.68 | 2.91 | 14 | w |
| 31.77 | 2.81 | 10 | w |
| 32.42 | 2.76 | 8 | w |
| 32.83 | 2.73 | 5 | vw |
| 33.28 | 2.69 | 7 | w |
| 34.34 | 2.61 | 8 | w |
| 35.50 | 2.53 | 6 | w |
| 35.93 | 2.50 | 7 | w |
| 37.40 | 2.40 | 4 | vw |
| 37.87 | 2.37 | 5 | vw |
| 38.59 | 2.33 | 7 | w |
| 39.49 | 2.28 | 5 | vw |

*vs = very strong,
s = strong,
m = medium,
w = weak,
vw = very weak

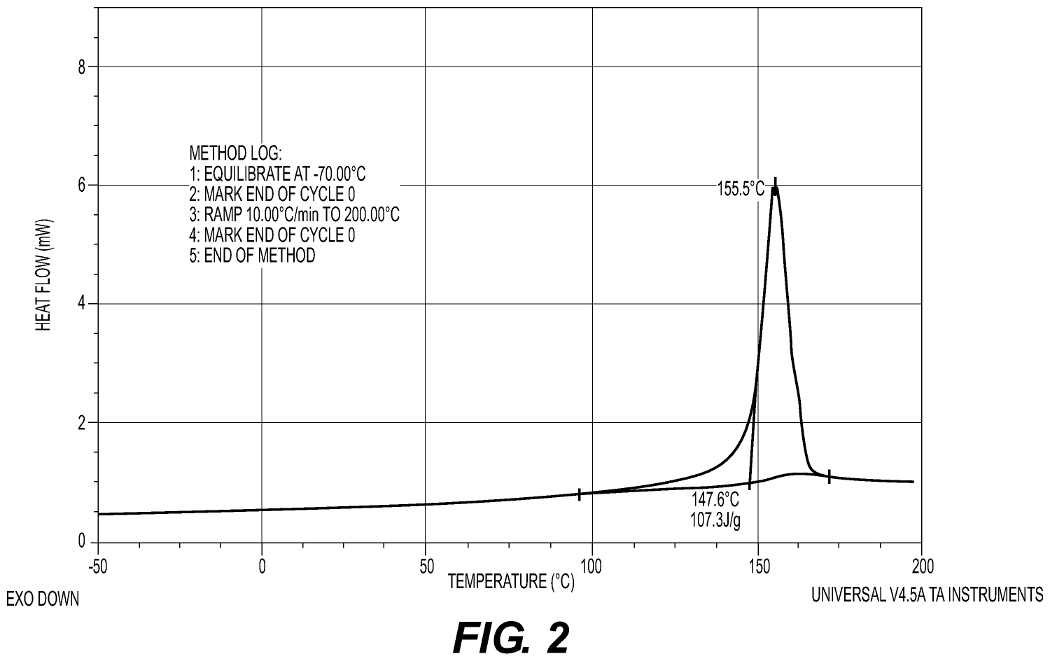

The DSC thermogram for crystalline polymorph form A is shown in FIG. 2.

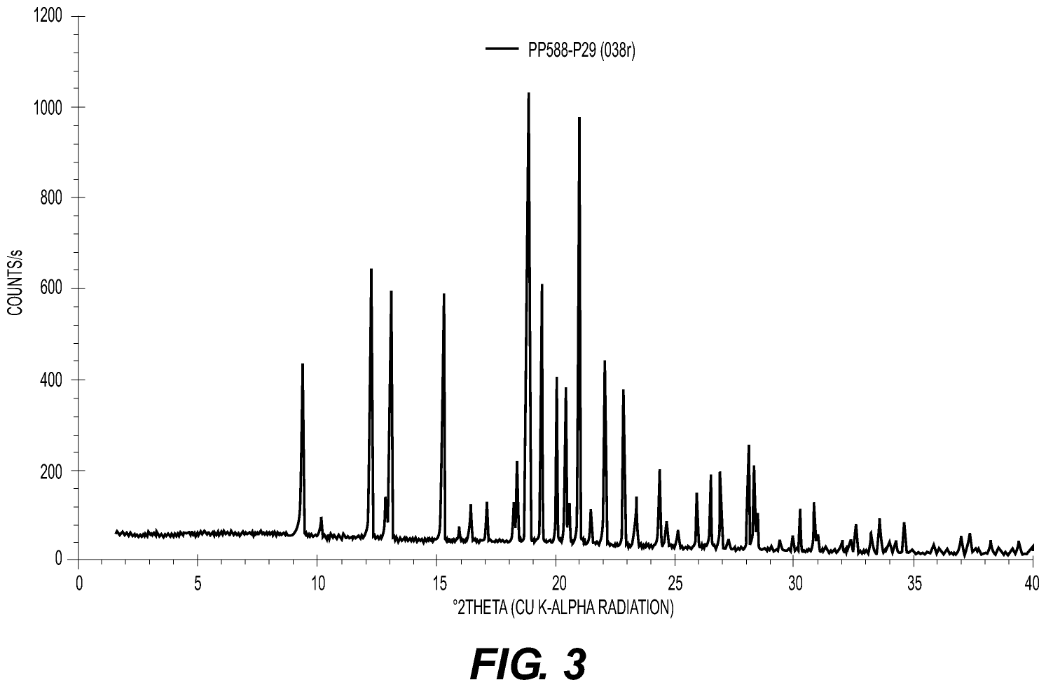

A unique physical form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one (solvent-free and anhydrous), polymorph form D, has been prepared according to the methods described herein. The powder X-ray diffraction (PXRD) pattern of free base polymorph form D is shown in FIG. 3, with corresponding tabulated data shown in Table 2.

TABLE 2

| | | Relative | Qualitative |
|---|---|---|---|
| °2-theta | d-spacing [Å] | intensity in % | intensity* |
| 9.38 | 9.4 | 43 | s |
| 10.16 | 8.7 | 10 | w |
| 12.26 | 7.2 | 63 | s |
| 12.86 | 6.9 | 14 | w |
| 13.08 | 6.8 | 58 | s |
| 15.27 | 5.80 | 58 | s |
| 15.94 | 5.56 | 8 | w |
| 16.41 | 5.40 | 13 | w |
| 17.09 | 5.18 | 13 | w |
| 18.22 | 4.87 | 13 | w |
| 18.38 | 4.82 | 21 | m |
| 18.84 | 4.71 | 100 | vs |
| 19.39 | 4.57 | 59 | s |
| 20.02 | 4.43 | 40 | s |
| 20.40 | 4.35 | 37 | s |
| 20.56 | 4.32 | 12 | w |
| 20.95 | 4.24 | 96 | vs |
| 21.45 | 4.14 | 11 | w |
| 22.02 | 4.03 | 43 | s |
| 22.84 | 3.89 | 36 | s |
| 23.33 | 3.81 | 14 | w |
| 24.31 | 3.66 | 20 | m |
| 24.62 | 3.61 | 9 | w |
| 25.09 | 3.55 | 7 | w |
| 25.88 | 3.44 | 15 | w |
| 26.48 | 3.36 | 19 | m |
| 26.88 | 3.31 | 19 | m |
| 27.21 | 3.28 | 5 | vw |
| 28.05 | 3.18 | 25 | m |
| 28.29 | 3.15 | 21 | m |
| 29.01 | 3.08 | 3 | vw |
| 29.37 | 3.04 | 5 | vw |
| 29.90 | 2.99 | 5 | w |
| 30.20 | 2.96 | 11 | w |
| 30.82 | 2.90 | 13 | w |
| 31.32 | 2.85 | 3 | vw |
| 31.95 | 2.80 | 4 | vw |
| 32.35 | 2.76 | 4 | vw |
| 32.56 | 2.75 | 8 | w |
| 33.20 | 2.70 | 6 | w |
| 33.54 | 2.67 | 9 | w |
| 33.98 | 2.64 | 4 | vw |
| 34.59 | 2.59 | 8 | w |
| 35.83 | 2.50 | 4 | vw |
| 36.96 | 2.43 | 5 | w |
| 37.33 | 2.41 | 6 | w |
| 38.20 | 2.35 | 5 | vw |
| 38.52 | 2.34 | 3 | vw |
| 39.08 | 2.30 | 3 | vw |
| 39.37 | 2.29 | 4 | vw |
| 39.96 | 2.25 | 3 | vw |

* vs = very strong,
s = strong,
m = medium,
w = weak,
vw = very weak

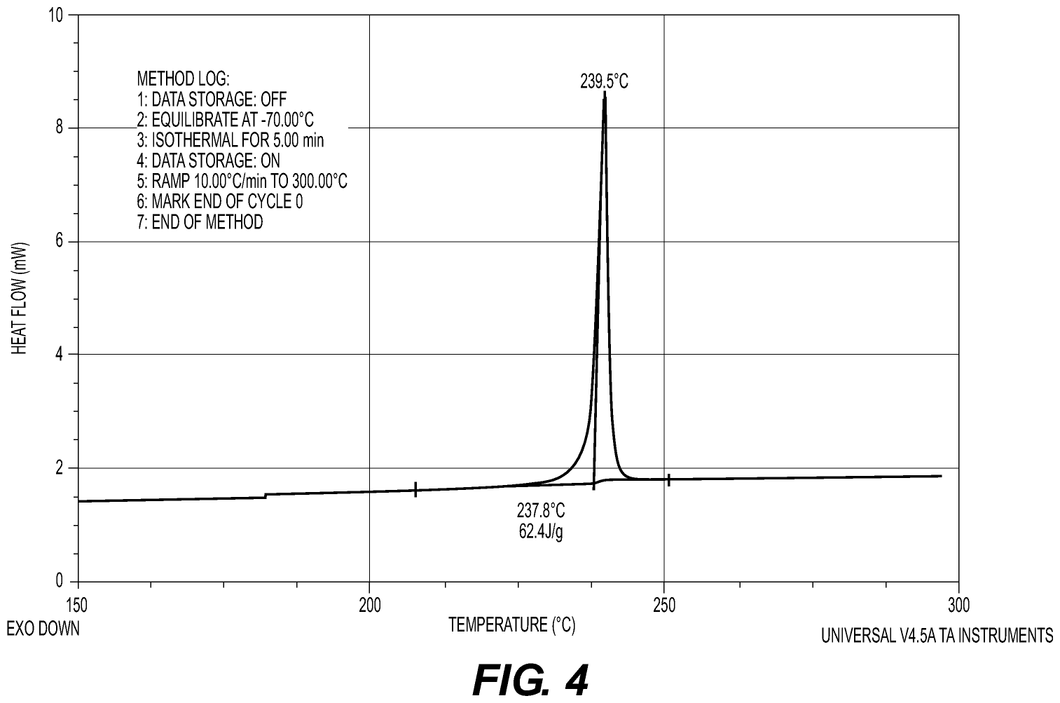

The DSC thermogram for crystalline polymorph form D is shown in FIG. 4.

In one aspect, the compounds and pharmaceutical compositions of the invention specifically target tyrosine receptor kinases, in particular RET, SRC or FGFR1/2. Thus, these compounds and pharmaceutical compositions can be used to prevent, reverse, slow, or inhibit the activity of one or more of these kinases. In some embodiments, methods of treatment disease mediated by one or more receptor tyrosine kinase are described herein.

Exemplary diseases include cancer, pain, neurological diseases, autoimmune diseases, and inflammation.

In some embodiments, methods of treating cancer are described herein comprising administering a therapeutically effective amount of a crystalline polymorph form A or D of Compound I. Cancer includes but is not limited to lung cancer, such as non-small cell lung cancer, small cell lung cancer, and the like, bone cancer, pancreatic cancer, skin cancer, cancer of the head or neck, hepatocellular carcinoma, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, gastric and esophago-gastric cancers, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, such as anaplastic large cell lymphoma, cancer of the bladder, cancer of the kidney or ureter, renal cell carcinoma, carcinoma of the renal pelvis, neoplasms of the central nervous system (CNS), such as glioblastoma, primary CNS lymphoma, spinal axis tumors, brain stem glioma, and the like, pituitary adenoma, inflammatory myofibroblastic tumors, and combinations thereof.

In some embodiments, the abnormal cell growth is mediated by RET, SRC, FGFR1/2 or a combination thereof. In some embodiments, the abnormal cell growth is mediated by wild-type or mutant RET. In some embodiments, the abnormal cell growth is mediated by wild-type or mutant SRC. In some embodiments, the abnormal cell growth is mediated by wild-type or mutant FGFR1/2.

In some embodiments, the present disclosure provides a method of treating abnormal cell growth in a patient in need of such treatment comprising, administering a therapeutically effective amount of Compound I. In some embodiments, the present disclosure provides a method of treating abnormal cell growth in a patient in need of such treatment comprising, administering a therapeutically effective amount of the free base polymorph form A or D of Compound I. In some embodiments, the abnormal cell growth is cancer mediated by a genetically altered RET. In some embodiments, the abnormal cell growth is cancer mediated by a genetically altered RET comprising at least one point mutation selected from the group consisting of A883F, E762Q, G691S, L790F, M918T, R749T, R813Q, S891A, S904A, S904F, V778I, V804L, V804M, Y791F, and Y806H.

In some embodiments, the present disclosure provides a method of treating abnormal cell growth in a patient in need of such treatment comprising, administering a therapeutically effective amount of Compound I. In some embodiments, the present disclosure provides a method of treating abnormal cell growth in a patient in need of such treatment comprising, administering a therapeutically effective amount of the free base polymorph form A or D of Compound I. In some embodiments, the abnormal cell growth is cancer mediated by a fusion protein comprising a fragment of a protein encoded by an RET gene and a fragment of a protein which can form coiled-coil interaction to facilitate the protein dimerization or oligomerization. In some embodiments, the abnormal cell growth is cancer mediated by a fusion protein comprising a fragment of a protein encoded by an RET gene and a fragment of a protein encoded by a gene selected from the group consisting of KIF5B, CCDC6, NCOA4, TRIM24, TRIM33, PRKAR1A, GOLGA5, KTN1, ERC1, MBD1, and TRIM27. In some embodiments, the fusion protein comprises a fragment of a protein encoded by an RET gene and a fragment of a protein encoded by a KIF5B gene. In some embodiments, the genetically altered RET is a KIF5B-RET, CCDC6-RET, NCOA4-RET, TRIM24-RET, TRIM33-RET, PRKAR1A-RET, GOLGA5-RET, KTN1-RET, ERC1-RET, MBD1-RET, or TRIM27-RET fusion protein. In some embodiments, the genetically altered RET is a KIF5B-RET fusion protein. In some embodiments, the KIF5B-RET fusion protein is a wild-type protein. In some embodiments, the KIF5B-RET fusion protein comprises at least one resistance mutation. In some embodiments, the KIF5B-RET fusion protein comprises at least one gate keeper resistance mutation. In some embodiments, the KIF5B-RET fusion protein comprises at least one solvent front resistance mutation. In some embodiments, the KIF5B-RET fusion protein comprises at least one mutation selected from the group consisting of A883F, E762Q, G691S, L790F, M918T, R749T, R813Q, S891A, S904A, S904F, V778I, V804L, V804M, Y791F, Y806H, V804M, G810R, G810C, and G810S. In some embodiments, the KIF5B-RET fusion protein comprises at least one mutation selected from the group consisting of V804M, G810R, G810C, and G810S.

In some embodiments, methods of treating or preventing pain are described herein comprising administering a therapeutically effective amount of a crystalline polymorph form A or D of Compound I. Pain includes, for example, pain from any source or etiology, including cancer pain, pain from chemotherapeutic treatment, nerve pain, pain from injury, or other sources.

In some embodiments, methods of treating autoimmune diseases are described herein comprising administering a therapeutically effective amount of a crystalline polymorph form A or D of Compound I. Autoimmune diseases include, for example, rheumatoid arthritis, Sjogren syndrome, Type I diabetes, and lupus. Exemplary neurological diseases include Alzheimer's Disease, Parkinson's Disease, Amyotrophic lateral sclerosis, and Huntington's disease.

In some embodiments, methods of treating inflammatory diseases of inflammation are described herein comprising administering a therapeutically effective amount of a crystalline polymorph form A or D of Compound I. Exemplary inflammatory diseases include atherosclerosis, allergy, and inflammation from infection or injury.

In treatment methods according to the invention, an “effective amount” means an amount or dose sufficient to generally bring about the desired therapeutic benefit in subjects needing such treatment. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Pharmaceutical Compositions

The present disclosure also relates to pharmaceutical compositions comprising the free base polymorph form A or D of Compound I described herein. Pharmaceutical compositions of the present disclosure may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition may include conventional pharmaceutically-acceptable excipients. In addition, pharmaceutical compositions described herein may include other medicinal or pharmaceutical agents, carriers, adjuvants, etc.

A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the invention are sterile compositions. Pharmaceutical compositions may be prepared using compounding techniques known or that become available to those skilled in the art.

Sterile compositions are also contemplated by the invention, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the invention may be administered by a suitable route of delivery, such as oral, parenteral, rectal, nasal, topical, or ocular routes, or by inhalation. In some embodiments, the compositions are formulated for intravenous or oral administration.

For oral administration, the compounds the invention may be provided in a solid form, such as a tablet or capsule, or as a solution, emulsion, or suspension. To prepare the oral compositions, the compounds of the invention may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present invention are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the invention may utilize a patch formulation to effect transdermal delivery

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present invention or may be included with a compound of the present invention in a single pharmaceutical composition. The additional active ingredients may be administered simultaneously with, prior to, or after administration of a compound of the present invention.

Combination agents include additional active ingredients are those that are known or discovered to be effective in treating the diseases and disorders described herein, including those active against another target associated with the disease. For example, compositions and formulations of the invention, as well as methods of treatment, can further comprise other drugs or pharmaceuticals, e.g., other active agents useful for treating or palliative for the target diseases or related symptoms or conditions. For cancer indications, additional such agents include, but are not limited to, kinase inhibitors, such as EGFR inhibitors (e.g., erlotinib, gefitinib), Raf inhibitors (e.g., vemurafenib), VEGFR inhibitors (e.g., sunitinib), ALK inhibitors (e.g., crizotinib) standard chemotherapy agents such as alkylating agents, antimetabolites, anti-tumor antibiotics, topoisomerase inhibitors, platinum drugs, mitotic inhibitors, antibodies, hormone therapies, or corticosteroids. For pain indications, suitable combination agents include anti-inflammatories such as NSAIDs. The pharmaceutical compositions of the invention may additional comprise one or more of such active agents, and methods of treatment may additionally comprise administering an effective amount of one or more of such active agents.

### EXAMPLES

The examples and preparations provided below further illustrate and exemplify particular aspects of embodiments of the disclosure. It is to be understood that the scope of the present disclosure is not limited in any way by the scope of the following examples.

### Abbreviations

The examples described herein use materials, including but not limited to, those described by the following abbreviations known to those skilled in the art:

| | |
|---|---|
| g | grams |
| eq | equivalents |
| mmol | millimoles |
| mol | moles |
| mL | milliliters |
| L | liters |
| EtOH | Ethanol |
| MeOH | Methanol |
| DCM | dichloromethane |
| MHz | megahertz |
| δ | chemical shift |
| THF | tetrahydrofuran |
| DMSO-d₆ | deuterated dimethyl sulfoxide |
| CDCl₃ | deuterated chloroform |
| t-BuOH | tert-butanol |
| DIEA or DIPEA or Hunig's Base | n,n-diisopropylethylamine |
| min or mins | minute or minutes |
| hrs, hr or h | hour or hours |
| TLC | thin layer chromatography |
| M | molar |
| FDPP | pentafluorophenyl diphenylphosphinate |
| DMF | N,N-dimethylformamide |
| KOt-Pent | Potassium tert-pentoxide |

### Example 1

### Synthesis of Compound I

Compound I was prepared according to the following synthetic scheme:

### Preparation of (3aR,11S,20a5)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one (5)

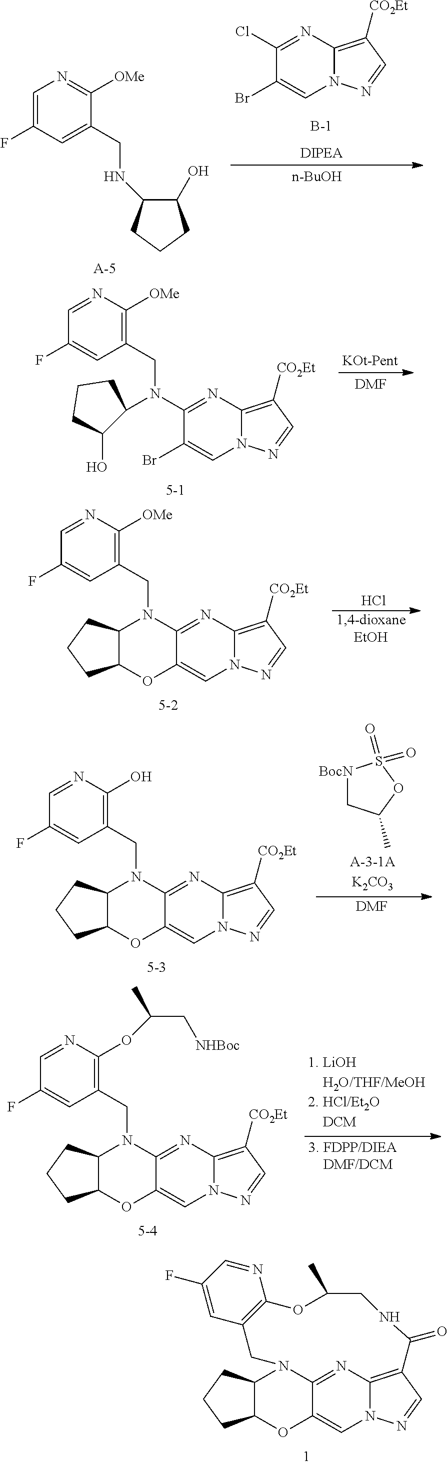

Step 1. To a solution of B-1 (454 mg, 1.49 mmol) and A-5 (358 mg, 1.49 mmol) in t-BuOH (5.0 mL) was added Hunig's base (963 mg, 7.45 mmol, 1.30 mL). The mixture was heated to 105° C. for 17 hr. The reaction was cooled and concentrated under reduced pressure. Flash chromatography (ISCO system, silica (12 g), 10-40% ethyl acetate in hexane) provided 5-1 (292 mg, 38% yield).

Step 2. To a solution of 5-1 (18.8 mg, 37 μmol) in DMF (3 mL) was added KOt-Pent (1.7 M, 65 μL) in toluene. The reaction stirred at room temperature for 20 hours. The reaction was cooled to −20° C. and quenched with saturated NH₄Cl sol. (5 mL) then extracted with DCM (3×10 mL). Combined extracts were dried with Na₂SO₄ and concentrated under reduced pressure. Flash chromatography (ISCO system, silica (12 g), 0-5% methanol in dichloromethane) provided 5-2 (6.2 mg, 39% yield).

Step 3. To a solution of 5-2 (6.2 mg, 14.5 μmol) in EtOH (4 mL) was added aqueous HCl solution (4.0 M, 3.0 mL) in 1,4-dioxane. The mixture was heated at 70° C. for 6 hours. The mixture was cooled, concentrated under reduced pressure, and dried under high vacuum to provide crude 5-3. Compound was used as is.

Step 4. Added K₂CO₃ (14.0 mg, 101 μmol) to 5-3 (6.2 mg, 14.5 μmol) and A-3-1A (17 mg, 73 μmol, tert-butyl (R)-5-methyl-1,2,3-oxathiazolidine-3-carboxylate 2,2-dioxide, available from e.g. Advanced ChemBlocks Inc) in DMF (250 μL) and stirred for 2 hours. The mixture was cooled and quenched with water (5 mL) then extracted with DCM (3×10 mL). Combined extracts were dried with Na₂SO₄ and concentrated under reduced pressure. Flash chromatography (ISCO system, silica (12 g), 20-100% ethyl acetate in hexane) provide 5-4 (6.1 mg, 73% yield).

Step 5. To a solution of 5-4 (6.1 mg, 10.7 μmol) in MeOH (3 mL) and THF (1 mL) at ambient temperature was added aqueous LiOH solution (2.0 M, 1.0 mL). The mixture was heated at 60° C. for 16 hr, cooled to −20° C. then quenched with aqueous HCl solution (2.0 M) to acidic. The mixture was extracted with DCM (3×5 mL), dried with Na₂SO₄. concentrated under reduced pressure, and dried under high vacuum. The crude material was dissolved in DCM (4 mL) followed by addition of HCl in 1,4-dioxane (4 M, 3 mL). The mixture was stirred at ambient temperature for 2 hr, concentrated under reduced pressure, and dried under high vacuum. The crude material was dissolved in in DMF (2.0 mL) and DCM (4.0 mL) and Hunig's base (185 mg, 1.4 mmol, 250 μL) then FDPP (34.5 mg, 89 μmol) was added in one portion. The reaction was stirred for 1 hr then quenched with 2 M Na₂CO₃ solution (5 mL). The mixture was stirred for 5 min then extracted with DCM (4×10 mL). Combined extracts were dried with Na₂SO₄ and concentrated under reduced pressure. Flash chromatography (ISCO system, silica (12 g), 0-5% methanol in dichloromethane) provided 5 (3.21 mg, 71% yield).

### Preparation of (1S,2R)-2-(((5-fluoro-2-methoxypyridin-3-yl)methyl)amino)cyclopentan-1-ol (A-5)

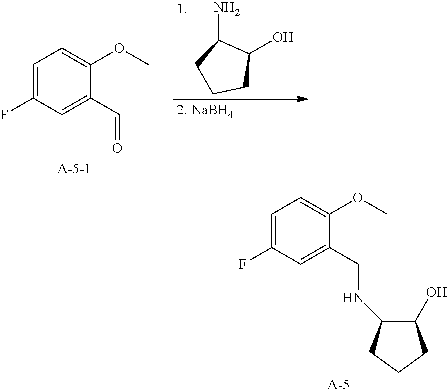 [SMILES: COc1ccc(F)cc1C=O ; N[C@@H]1CCC[C@@H]1O ; COc1ccc(F)cc1CN[C@@H]1CCC[C@@H]1O ; "A-5"]

A solution of (1S,2R)-2-aminocyclopentanol HCl salt (69 mg, 504 μmol), Hunig's Base (196 mg, 0.26 mL, 1.5 mmol) and A-1-4 (150.00 mg, 504 μmol) in dry MeOH (2.50 mL) was heated to 65° C. for 1 hr. The reaction was cooled to room temperature and NaBH₄ (38 mg, 1.0 mmol) was added. The mixture was stirred for 2 hr then quenched with water (3 mL) and stirred for 5 min. The mixture was extracted with DCM (3×5 mL), dried with Na₂SO₄ and concentrated under reduced pressure. Flash chromatography (ISCO system, silica (12 g), 25-50% ethyl acetate in hexane) provided A-1 (125.3 mg, 327 μmol, 64.9% yield).

Compound A-5 was prepared according to General Method A using 5-Fluoro-2-methoxynicotinaldehyde in step 2.

### Preparation of ethyl 6-bromo-5-chloropyrazolo[1,5-a]pyrimidine-3-carboxylate (B-1)

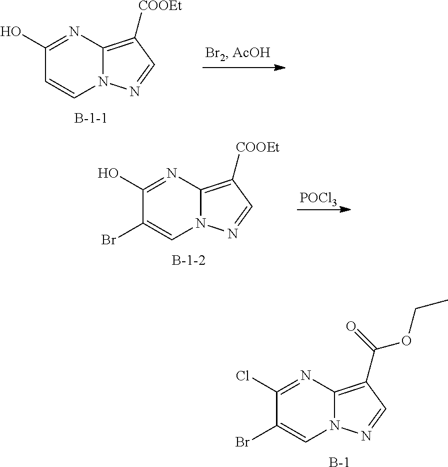

Step 1. To a solution of B-1-1 (10.00 g, 47.80 mmol, 1.00 eq., ethyl 5-hydroxypyrazolo[1,5-a]pyrimidine-3-carboxylate, available from e.g. Suzhou Devi Pharma Technology Co, Ltd.) in acetic acid (100.00 mL) was added bromine (7.64 g, 47.80 mmol, 2.46 mL, 1.00 eq.). The mixture was stirred at 180° C. for 6 hr. TLC (petroleum ether/ethyl acetate=1/1) showed the starting material was consumed completely and one new spot was found. The mixture was quenched by water (30 mL). The mixture was filtered and the cake was concentrated to give B-1-2 (10.00 g, 34.71 mmol, 72.62% yield) as a white solid: ¹H NMR (400 MHz, DMSO-d6) δ: 12.34 (br. s., 1H), 9.25 (s, 1H), 8.15 (s, 1H), 4.28 (q, J=7.2 Hz, 2H), 1.29 (t, J=7.2 Hz, 3H).

Step 2. To a solution of B-1-2 (6.00 g, 20.97 mmol, 1.00 eq.) in phosphorus oxychloride (60 mL). The mixture was stirred at 120° C. for 16 hr. TLC (petroleum ether/ethyl acetate=3/1) indicated the starting material was consumed completely and one new spot was found. The reaction mixture was filtered and concentrated under reduced pressure to give a residue. The residue was purified by column chromatography (SiO₂, petroleum ether/ethyl acetate=10/1 to 1/1) to give B-1 (2.50 g, 8.21 mmol, 39.15% yield) as a white solid; ¹H NMR (400 MHz, CDCl₃) δ: 8.94 (s, 1H), 8.54 (s, 1H), 4.43 (q, J=7.2 Hz, 2H), 1.42 (t, J=7.2 Hz, 3H).

### Example 2

Formation of Crystalline Polymorph Form A of Compound I.

A solution of Compound I (13.8 g) in ethanol (30 mL) was treated with water (10 mL). Solid Compound I, obtained directly from the precipitation, was crystalline polymorph form A of Compound I.

### Example 3

Formation of Crystalline Polymorph Form D of Compound I.

Solid Compound I crystalline polymorph form A (302 mg), obtained directly from the purification fractions of Example 1, Step 5, was equilibrated in anhydrous 2-propanol (1.5 mL) to provide a crystalline polymorph form D of Compound I.

Testing of Crystalline Polymorph Form A of Compound I

### Example 4

Powder X-Ray Diffraction (PXRD) of Crystalline Polymorph Form A of Compound I.

A sample of Compound I, crystalline polymorph form A, was run for PXRD on a Stoe Stadi P powder X-ray diffractometer equipped with a Mythen1K Detector and Cu-Kα1 radiation. The sample was spun during collection to limit preferred orientation peaks. Data was collected from 1.5°-50.5° 2θ using a step size of 0.02° 2θ and scan rate of 12 s per step. Results are shown in FIG. 1.

### Example 5

Differential Scanning Calorimetry (DSC) of Crystalline Polymorph Form A of Compound I.

DSC measurement was carried out with a TA Instruments DSC Q2000 using hermetically sealed gold sample pans. A 3.2150 mg sample of Compound I, crystalline polymorph form A, was equilibrated at −70° C., and then ramped to 200° C. at a rate of 10° C./min. The sample of Compound I, crystalline polymorph form A showed an endothermal peak with a peak temperature of 155.5° C. Results are shown in FIG. 2.

### Example 6

FT-Raman Spectrum of Crystalline Polymorph Form A of Compound I.

FT-Raman was carried out on a sample of Compound I, crystalline polymorph form A using a Bruker MultiRAM FT-Raman system with a near infrared Nd:YAG laser operating at 1064 nm and a liquid nitrogen-cooled germanium detector. 64 scans with a resolution of 2 cm⁻¹ were accumulated in the range from 3500 to 50 cm⁻¹. Laser power were 300 mW. Only data above 100 cm⁻¹ were evaluated due to filter cutoff effects. The sample of Compound I, crystalline polymorph form A, showed absorptions as in Table 3:

TABLE 3

| Wavenumber cm⁻¹ | arbitrary Raman intensity |
|---|---|
| 3093 | 0.70 |
| 3060 | 0.27 |
| 2993 | 0.72 |
| 2968 | 1.05 |
| 2945 | 1.16 |
| 2922 | 1.02 |
| 2870 | 0.55 |
| 1643 | 1.30 |
| 1594 | 0.32 |
| 1567 | 0.54 |
| 1519 | 1.58 |
| 1488 | 1.21 |
| 1456 | 0.45 |
| 1435 | 0.97 |
| 1395 | 1.46 |
| 1382 | 1.79 |
| 1359 | 1.22 |
| 1352 | 1.05 |
| 1329 | 2.27 |
| 1277 | 0.81 |
| 1244 | 0.46 |
| 1200 | 0.29 |
| 1120 | 0.39 |
| 1103 | 0.29 |
| 1052 | 0.28 |
| 1031 | 0.22 |
| 993 | 0.23 |
| 956 | 0.15 |
| 936 | 0.20 |
| 903 | 0.45 |
| 896 | 0.40 |
| 876 | 0.31 |
| 797 | 0.30 |
| 778 | 0.32 |
| 745 | 0.21 |
| 719 | 0.59 |
| 584 | 0.22 |
| 567 | 0.17 |
| 524 | 0.32 |
| 451 | 0.25 |
| 428 | 0.25 |
| 414 | 0.40 |
| 387 | 0.39 |
| 366 | 0.48 |
| 351 | 0.26 |
| 325 | 0.39 |
| 274 | 0.85 |
| 171 | 0.76 |
| 155 | 0.57 |
| 133 | 0.79 |

Testing of Crystalline Polymorph Form D of Compound I

### Example 7

Powder X-Ray Diffraction (PXRD) of Crystalline Polymorph Form D of Compound I.

A sample of Compound I, crystalline polymorph form D, was run for PXRD on a Stoe Stadi P powder X-ray diffractometer equipped with a Mythen1K Detector and Cu-Kα1 radiation. The sample was spun during collection to limit preferred orientation peaks. Data was collected from 1.5°-50.5° 2θ using a step size of 0.02° 2θ and scan rate of 12 s per step. Results are shown in FIG. 3.

### Example 8

Differential Scanning Calorimetry (DSC) of Crystalline Polymorph Form D of Compound I.

DSC measurement was carried out with a TA Instruments DSC Q2000 using hermetically sealed gold sample pans. A 1.8860 mg sample of Compound I, crystalline polymorph form D, was equilibrated at −70° C., and then ramped to 300° C. at a rate of 10° C./min. The sample of Compound I, crystalline polymorph form D showed an endothermal peak with a peak temperature of 239.5° C. Results are shown in FIG. 4.

### Biological Examples

### Example 9

Biochemical Kinase Assay Method

The biochemical kinase assay was performed at Reaction Biology Corporation (www.reactionbiology.com, Malvern, Pa.) following the procedures described in the reference (Anastassiadis T, et al Nat Biotechnol. 2011, 29, 1039). Specific kinase/substrate pairs along with required cofactors were prepared in reaction buffer; 20 mM Hepes pH 7.5, 10 mM MgCl₂, 1 mM EGTA, 0.02% Brij35, 0.02 mg/ml BSA, 0.1 mM Na₃VO₄, 2 mM DTT, 1% DMSO. Compounds were delivered into the reaction, followed ˜20 minutes later by addition of a mixture of ATP (Sigma, St. Louis Mo.) and ³³P ATP (Perkin Elmer, Waltham Mass.) to a final concentration of 10 μM. Reactions were carried out at room temperature for 120 min, followed by spotting of the reactions onto P81 ion exchange filter paper (Whatman Inc., Piscataway, N.J.). Unbound phosphate was removed by extensive washing of filters in 0.75% phosphoric acid. After subtraction of background derived from control reactions containing inactive enzyme, kinase activity data was expressed as the percent remaining kinase activity in test samples compared to vehicle (dimethyl sulfoxide) reactions. IC₅₀ values and curve fits were obtained using Prism (GraphPad Software).

Cell Lines and Cell Culture:

Human medulla thyroid carcinoma cell line TT (containing RET M918T mutation) and acute myelogenous cell line KG-1 were purchased from ATCC. Human colon cancer cell line KM12 (containing TPM3-TRKA) was obtained from NCI.

Cloning and Ba/F3 Stable Cell Line Creation

The EML4-ALK gene (variant 1) was synthesized at GenScript and cloned into pCDH-CMV-MCS-EF1-Puro plasmid (System Biosciences, Inc). Ba/F3-EML4-ALK wild type were generated by transducing Ba/F3 cells with lentivirus containing EML4-ALK wide type. Stable cell lines were selected by puromycin treatment, followed by IL-3 withdrawal. Briefly, 5×10⁶ Ba/F3 cells were transduced with lentivirus supernatant in the presence of 8 μg/mL protamine sulfate. The transduced cells were subsequently selected with 1 μg/mL puromycin in the presence of IL3-containing medium RPMI1640, plus 10% FBS. After 10-12 days of selection, the surviving cells were further selected for IL3 independent growth.

The KIF5B-RET gene was synthesized at GenScript and cloned into pCDH-CMV-MCS-EF1-Puro plasmid (System Biosciences, Inc). KIF5B-RET point mutation V804M and G810C were generated at GenScript by PCR and confirmed by sequencing. Ba/F3-KIF5B-RET wild type and mutant were generated by transducing Ba/F3 cells with lentivirus containing KIF5B-RET wide type or mutant. Stable cell lines were selected by puromycin treatment, followed by IL-3 withdrawal. Briefly, 5×10⁶ Ba/F3 cells were transduced with lentivirus supernatant in the presence of 8 μg/mL protamine sulfate. The transduced cells were subsequently selected with 1 μg/mL puromycin in the presence of IL3-containing medium RPMI1640, plus 10% FBS. After 10-12 days of selection, the surviving cells were further selected for 113 independent growth.

Cell Proliferation Assays:

Two thousand cells per well were seeded in 384 well white plate for 24 hrs, and then treated with compounds for 72 hours (37° C., 5% CO₂). Cell proliferation was measured using CellTiter-Glo luciferase-based ATP detection assay (Promega) following the manufactures's protocol. IC₅₀ determinations were performed using GraphPad Prism software (GraphPad, Inc., San Diego, Calif.).

Data and Results:

Enzymatic Kinase Activities of Compound I.

| Cpd I | IC₅₀ (nM) at 10 μM ATP |
|---|---|
| RET | 1.0 |
| RET (A883F) | 1.2 |
| RET (E762Q) | 1.0 |
| RET (G691S) | 1.8 |
| RET (L790F) | 0.4 |
| RET (M918T) | 0.3 |
| RET (R749T) | 0.8 |
| RET (R813Q) | 1.7 |
| RET (S891A) | 0.2 |
| RET (S904A) | 0.9 |
| RET (S904F) | 0.5 |
| RET (V778I) | 0.1 |
| RET (V804E) | >1000 |
| RET (V804L) | 6.2 |
| RET (V804M) | 8.1 |
| RET (Y791F) | 0.4 |
| RET (Y806H) | 2.6 |
| RET-CCDC6 (PTC1) | 0.5 |
| RET-NCOA4 (PTC3) | 0.7 |
| SRC | 1.5 |
| FYN | 1.9 |
| LYN | 2.0 |
| YES/YES1 | 2.6 |
| HCK | 2.7 |

Anti-Cell Proliferation Activity

| | | | | | Ba/F3 | KM12 | Ba/F3 |
|---|---|---|---|---|---|---|---|
| | | | TT cell | Ba/F3 | KIF5B- | cell | KIF5B- |
| | | | (RET | KIF5B- | RET_ | (TPM3- | RET_ |
| | Ba/F3 | KG-1 | M918T) | RET | V804M | TRKA) | G810C |
| | EML4- | IC₅₀ | IC₅₀ | IC₅₀ | IC₅₀ | IC₅₀ | IC₅₀ |
| Cpd | ALK | (nM) | (nM) | (nM) | (nM) | (nM) | (nM) |
| 1 | 211.4 | 131.2 | <0.5 | 0.25 | 1002 | 3.0 | <15 |

Compound I Anti-Cell Proliferation Comparison

| | Experiment 1 | | | Experiment 2 | | |
|---|---|---|---|---|---|---|
| | IC₅₀ | IC₇₅ | IC₉₀ | IC₅₀ | IC₇₅ | IC₉₀ |
| CPD | (nM) | (nM) | (nM) | (nM) | (nM) | (nM) |
| LOXO- | 301.3 | 1021 | 3447 | 363.8 | 791.4 | 2374 |
| 292 | | | | | | |
| BLU-667 | 18.5 | 115.4 | 720.4 | 44.6 | 214.3 | 1029 |
| Cpd I | 9.6 | 28.8 | 86.4 | 13.3 | 39.9 | 119.7 |

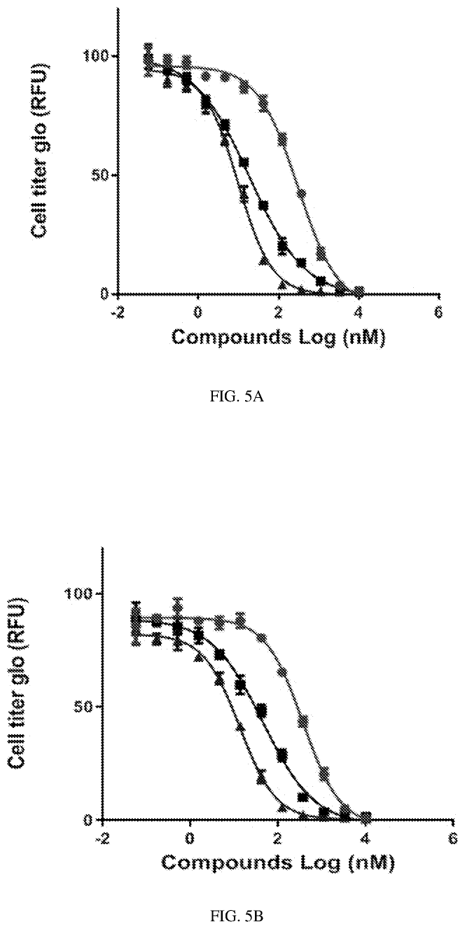

See also FIG. 5A and FIG. 5B.

Compound I Potently Inhibits Ba/F3 KIF5B-RET G810C Autophosphorylation

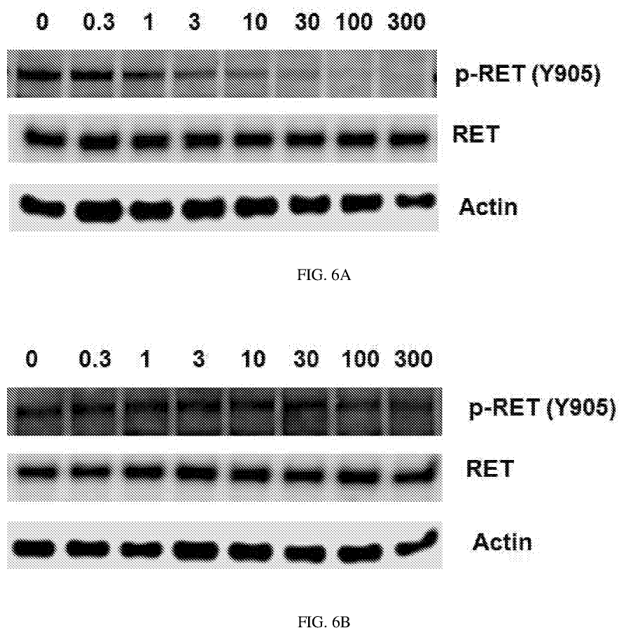

Effect of Compound I and LOXO-292 on RET-G810C phosphorylation at residue Y905 in Ba/F3 engineered cells. Various concentrations of Compound I, LOXO-292 were incubated with Ba/F3 KIF5B-RET G810C (1 million cells per well in 24 well plate) for 4 hours. Cells were collected, lysed, and p-RET (Y905, RET level were determined by SDS-PAGE and immunoblotting using related antibodies. Actin was used as a loading control. Compound I had an IC₅₀ between 1-3 nM while LOXO-292 was greater than 300 nM. Results are shown in FIG. 6A and FIG. 6B.

1. A crystalline polymorph form of (3aR,11S,20aS)-7-fluoro-11-methyl-2,3,3a,12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one, or a hydrate thereof.

2. The crystalline polymorph form of claim 1, wherein the crystalline form is a polymorph form of the free base of (3aR,11S,20aS)-7-fluoro-11-methyl-2,3,3a12,13,20a-hexahydro-1H,5H-17,19-(metheno)cyclopenta[5,6][1,4]oxazino[3,4-i]pyrazolo[4,3-f]pyrido[3,2-l][1,4,8,10]oxatriazacyclotridecin-14(11H)-one, or a hydrate thereof.

3. The crystalline polymorph of claim 1, wherein the crystalline form is a monohydrate.

4. The crystalline polymorph of claim 1, wherein the crystalline form is anhydrous.

5. The crystalline polymorph form of claim 1, having a powder X-ray diffraction pattern comprising a peak at diffraction angle (2θ) of 20.1±0.1.

6. The crystalline polymorph form of claim 5, wherein the crystalline polymorph form has a powder X-ray diffraction pattern comprising a peak at diffraction angle (2θ) of 11.3±0.1 and 20.1±0.1.

7. The crystalline polymorph form of claim 5, wherein the crystalline polymorph form has a powder X-ray diffraction pattern comprising a peak at diffraction angle (2θ) of 11.3±0.1, 20.1±0.1, and 23.9±0.1.

8. The crystalline polymorph form of claim 5 wherein the crystalline polymorph form has a powder X-ray diffraction pattern comprising a peak at diffraction angle (2θ) of 11.3±0.1, 18.0±0.1, 20.1±0.1, and 23.9±0.1.

9. The crystalline polymorph form of claim 5, wherein the crystalline polymorph form has a powder X-ray diffraction pattern comprising a peak at diffraction angle (2θ) of 10.3±0.1, 11.3±0.1, 18.0±0.1, 20.1±0.1, and 23.9±0.1.

10. The crystalline polymorph form of claim 5, wherein the crystalline polymorph form has a powder X-ray diffraction pattern comprising a peak at diffraction angle (2θ) of 10.3±0.1, 11.3±0.1, 16.6±0.1, 18.0±0.1, 20.1±0.1, and 23.9±0.1.

11. The crystalline polymorph form of claim 5, wherein the crystalline polymorph form has a powder X-ray diffraction pattern comprising a peak at diffraction angle (2θ) of 10.3±0.1, 11.3±0.1, 16.6±0.1, 18.0±0.1, 20.1±0.1, 20.5±0.1, and 23.9±0.1.

12. The crystalline polymorph form of claim 1 having a powder X-ray diffraction pattern comprising a peak at diffraction angle (2θ) of 18.8±0.1.

13. The crystalline polymorph form of claim 12, wherein the crystalline polymorph form has a powder X-ray diffraction pattern comprising a peak at diffraction angle (2θ) of 18.8±0.1 and 20.9±0.1.

14. The crystalline polymorph form of claim 12, wherein the crystalline polymorph form has a powder X-ray diffraction pattern comprising a peak at diffraction angle (2θ) of 12.3±0.1, 18.8±0.1, and 20.9±0.1.

15. The crystalline polymorph form of claim 12, wherein the crystalline polymorph form has a powder X-ray diffraction pattern comprising a peak at diffraction angle (2θ) of 12.3±0.1, 18.8±0.1, 19.4±0.1, and 20.9±0.1.

16. The crystalline polymorph form of claim 12, wherein the crystalline polymorph form has a powder X-ray diffraction pattern comprising a peak at diffraction angle (2θ) of 12.3±0.1, 13.1±0.1, 18.8±0.1, 19.4±0.1, and 20.9±0.1.

17. The crystalline polymorph form of claim 12, wherein the crystalline polymorph form has a powder X-ray diffraction pattern comprising a peak at diffraction angle (2θ) of 12.3±0.1, 13.1±0.1, 15.3±0.1, 18.8±0.1, 19.4±0.1, and 20.9±0.1.

18. The crystalline polymorph form of claim 12, wherein the crystalline polymorph form has a powder X-ray diffraction pattern comprising a peak at diffraction angle (2θ) of 9.4±0.1, 12.3±0.1, 13.1±0.1, 15.3±0.1, 18.8±0.1, 19.4±0.1, and 20.9±0.1.

19. The crystalline polymorph form of claim 1, having a Raman spectra comprising at least one, at least two, at least three, at least four, at least five, at least six, at least seven, or at least about 8 peaks selected from Table 3.

20. The crystalline polymorph form of claim 1, having a powder X-ray diffraction pattern substantially the same as shown in FIG. 1.

21. The crystalline polymorph form of claim 1, having a powder X-ray diffraction pattern substantially the same as shown in FIG. 3.

22. A pharmaceutical composition comprising the crystalline polymorph form of claim 1.

23. A method of treating cancer in a human in need thereof, the method comprising administering to the human a therapeutically effective amount of the crystalline polymorph form of claim 1.

24.-26. (canceled)

27. The method of claim 2, wherein the cancer is selected from the groups consisting of lung cancer, non-small cell lung cancer, small cell lung cancer, bone cancer, pancreatic cancer, skin cancer, cancer of the head or neck, hepatocellular carcinoma, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, gastric and esophago-gastric cancers, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, such as anaplastic large cell lymphoma, cancer of the bladder, cancer of the kidney or ureter, renal cell carcinoma, carcinoma of the renal pelvis, neoplasms of the central nervous system (CNS), glioblastoma, primary CNS lymphoma, spinal axis tumors, brain stem glioma, pituitary adenoma, inflammatory myofibroblastic tumors, and combinations thereof.

28.-40. (canceled)
